(19) United States
(12) Patent Application Publication
(10) Pub. No.: US 2003/0152930 A1
(43) Pub. Date: Aug. 14, 2003

(54) Method and apparatus for detecting DNA hybridization

(75) Inventor: John K. Howard, Saratoga, CA (US)

Correspondence Address: McDERMOTT, WILL & EMERY, 600 13th Street, N.W., Washington, DC, 20005-3096, US

(73) Assignee: Matsushita Electric Industrial Co., Ltd.

(21) Appl. No.: 10/068,854

(22) Filed: Feb. 11, 2002

(51) Int. Cl. C12Q 1/68; C12M 1/34

(52) U.S. Cl. 435/6; 435/287.2; 205/777.5

(57) ABSTRACT

An apparatus for identifying an unknown DNA sample. The apparatus includes a plurality of detection nodes, each of which is operable for allowing an interaction between a known DNA sample and an unknown DNA sample, and for generating an output signal if hybridization occurs between the known DNA sample and the unknown DNA sample. The apparatus further includes a decoder operative for receiving an input signal indicative of which of the plurality of detection nodes should be selected for processing and for outputting control signals which operate to activate the selected detection node. Further, each of the detection nodes comprises a first capacitor having a capacitance value which varies if hybridization occurs between the known DNA sample and the unknown DNA sample contained in the first capacitor. This change of capacitance is utilized to generate the output signal which indicates that hybridization has occurred.

### FIELD OF THE INVENTION

This invention relates generally to a system and method of detecting interactions between analyte molecules, and more particularly, to a system and method for determining DNA hybridization.

### BACKGROUND OF THE INVENTION

DNA sequencing is becoming a major factor in a number of different emerging scientific fields. For example, DNA sequencing has been utilized in an attempt to diagnosis various diseases. One known method of performing such DNA sequencing/analysis is by matching an array of known DNA sequences (referred to as probes) with an unknown target DNA. More specifically, such a process typically includes placing a number of known DNA sequences on a glass slide. Each of the known DNA sequences are placed at a specific geographic location on the glass slide. A typical glass slide may have the capability of containing 50,000 individual locations, thereby allowing for processing of 50,000 DNA sequences.

Once the known DNA probes are placed in the predetermined locations on the slide, an unknown sample of DNA is placed on the entire slide. After a set period of time, if the unknown sample of DNA matches any of the known DNA sequences, the unknown DNA sample will hybridize with the known DNA sequence at the given location of the known DNA sequence. Assuming there is a match, the unknown sample DNA is identified as the DNA sequence with which the hybridization occurred.

In accordance with the foregoing technique, it is necessary to determine whether hybridization has occurred as well as the specific location of the hybridization so as to allow for a correlation between the location of the hybridization and the corresponding known DNA sequence. One common method of performing this determination is by an optical detection technique. In accordance with this technique, first, after allowing sufficient time for hybridization between the known DNA sequences and the unknown sample DNA, the slide is treated such that all un-hybridized DNA are removed from the slide. Next, an optical detection technique is utilized to determine the presence of a fluorescent molecule, which is attached to each known DNA sequence prior to the hybridization process. Specifically, if hybridization has occurred, the fluorescent molecule (i.e., die) attached to the known DNA sequence will be present even after the known DNA sequence has hybridized with the unknown DNA sample (if there was no match, all the known DNA sequences along with the fluorescent molecule would be removed from the slide during the aforementioned treatment process). Accordingly, by utilizing, for example, a laser and a photo detector, it is possible to determine the presence and location of the fluorescent molecule, which identifies the sample DNA by correlating the position of the fluorescent molecule with the location of the known DNA sequences. Typically, the instrument utilized to determine the presence of the fluorescent molecule is a desktop micro-array scanner.

Specific examples of such known optical detection systems and methods are set forth in U.S. Pat. No. 5,578,832, “Method And Apparatus For Imaging A Sample On A Device” issued to Trulson et al., and U.S. Pat. No. 5,631,734, “Method And Apparatus For Detection Of Fluorescently Labeled Materials”, issued to Stern et al. Both of the foregoing patents are hereby incorporated by reference. Utilizing a method similar to that described above, both of the foregoing patents employ the use of a fluorescent molecule, such as fluorophore and biotin, which is attached to the known DNA sequence. An optical system is then utilized to determine whether hybridization has occurred by measuring fluorescence activated between the sample DNA and the known DNA.

Another known technique for identifying unknown DNA sequences is disclosed in U.S. Pat. No. 6,203,983, which is also hereby incorporated by reference. As disclosed therein, a method is presented which allows for the detection of a chemical interaction (e.g., DNA hybridization) without having to modify (i.e., label) the known DNA sequence. Specifically, the method entails formation of a mechanical cantilever mechanism capable of physical movement in the upward and downward direction. The cantilever mechanism is arranged in conjunction with the sample DNA and known DNA such that hybridization o f the DNA will result in the physical deflection of the cantilever, which can be detected, thereby allowing for identification of the sample DNA.

Notwithstanding the foregoing chemical interaction detection systems utilized to identify unknown DNA samples, problems remain. For example, the systems utilizing optical detection means to detect fluorescent markers can be expensive. Moreover, the time requirements for operating the system can be exceedingly long as a typical array to be analyzed may contain on the order of 50,000 DNA samples, which need to be scanned on a one-by-one basis during processing. Systems utilizing micromechanical devices, such as the cantilever mechanism disclosed in the '983 patent, require elaborate semiconductor processing techniques during the formation thereof, which increase the costs associated with the resulting test arrays. Moreover, such devices are exceedingly subject to failure due to mechanical nature of the operation of the array, thereby reducing the overall reliability of the resulting array.

Accordingly, there remains a need for providing a detection system capable of identifying unknown DNA samples that eliminates the need for the optical scanner so as to allow for a reduction in both the time and cost associated with performing the analysis, and a reduction of the size of the equipment necessary for performing the analysis so as to facilitate home and clinical use. In addition, it is desirable that the detection system eliminate the need for micromechanical devices so as to improve the overall reliability of the system.

It is the object of the present invention to correct the foregoing deficiencies in the prior art.

### SUMMARY OF THE INVENTION

In general, the present invention relates to a DNA detection system that provides for identification of the unknown DNA in an electronic manner. The DNA detection system of the present invention eliminates the need for utilizing an optical scanner during the detection process, and the elimination of micromechanical devices from the detection system.

In a first exemplary embodiment, the present invention relates to an apparatus for identifying an unknown DNA sample. The apparatus comprises a plurality of detection nodes, each of which is operable for allowing an interaction between a known DNA sample and an unknown DNA sample, and for generating an output signal if hybridization occurs between the known DNA sample and the unknown DNA sample. The apparatus further comprises a decoder operative for receiving an input signal indicative of which of the plurality of detection nodes should be selected for processing and for outputting control signals which operate to activate the selected detection node. Further, each of the detection nodes comprises a first capacitor having a capacitance value which varies if hybridization occurs between the known DNA sample and the unknown DNA sample contained in the first capacitor. This change of capacitance is utilized to generate the output signal which indicates that hybridization has occurred.

The present invention also relates to a method of identifying a unknown DNA sample. The method comprises the steps of generating a first reference voltage signal; generating a second reference voltage signal utilizing a first capacitor having a known DNA sample disposed therein and which is capable of receiving the unknown DNA sample, where the first capacitor has a capacitance value that varies if hybridization occurs between the known DNA sample and the unknown DNA sample when the unknown DNA sample is delivered to the first capacitor; and generating an output signal representing a difference between the first reference voltage signal and the second reference voltage signal.

As described in further detail below, the present invention provides significant advantages over the prior art. Most importantly, the method and system of detecting/identifying unknown DNA of the present invention allows for the elimination of the need for utilizing an optical scanner during the detection process, and allows for real-time detection of unknown DNA. As such, the present invention allows for a reduction in the overall cost and time associated with performing the detection analysis.

Another advantage of the present invention is that it eliminates the need for utilizing micromechanical devices in the detection system, thereby increasing the overall reliability of the detection system. Yet another advantage is that the present invention eliminates the requirement of placing an optical florescent tag on the unknown DNA, thereby saving time and cost.

Additional advantages of the present invention will become apparent to those skilled in the art from the following detailed description of exemplary embodiments of the present invention.

The invention itself, together with further objects and advantages, can be better understood by reference to the following detailed description and the accompanying drawings.

### BRIEF DESCRIPTION OF THE DRAWINGS

FIG. 1 is a block diagram of an exemplary embodiment of the DNA detection system in accordance with the present invention.

FIG. 2 is an exemplary schematic diagram illustrating one embodiment of a detection node utilized on the DNA detection system illustrated in FIG. 1.

FIGS. 3a and 3b illustrate an exemplary embodiment of capacitors utilized in the detection node illustrated in FIG. 2.

FIG. 4 is an exemplary schematic diagram illustrating another embodiment of a detection node utilized on the DNA detection system illustrated in FIG. 1.

### DETAILED DESCRIPTION OF THE DRAWINGS

The following detailed description relates to a novel DNA detection system that allows for electronic detection/identification of unknown DNA. The description of the exemplary embodiment of the system sets forth numerous specific details regarding the configuration of the system. It will be obvious, however, to one skilled in the art that these specific details need not be employed to practice the present invention. Clearly, other configurations and implementations of the DNA detection system are possible.

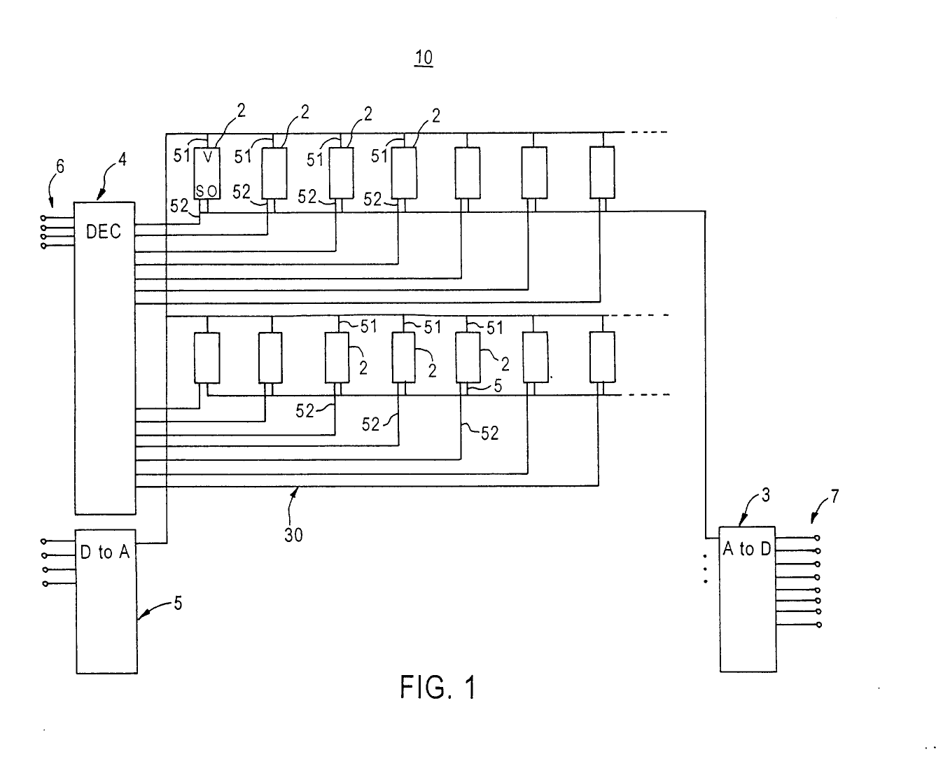

FIG. 1 illustrates a block diagram of an exemplary embodiment of the DNA detection system 10 in accordance with the present invention. Referring to FIG. 1, the system 10 comprises a plurality of addressable detection nodes 2, a node selection decoder 4, a D/A converter 5 and an A/D converter 3. As explained in further detail below, each of the plurality of detection nodes 2 have an identical structure and is capable of comparing a known DNA sample with an unknown DNA and outputting an electronic signal if the given DNA sample and unknown DNA hybridize (i.e., match). Each addressable detection node 2 comprises three signal lines coupled thereto. The first is a variable input voltage signal line 51 for receiving a voltage signal output by D/A converter 5. A variable voltage source (e.g, a digital voltage signal generated by a computer controller) is coupled to the input of the D/A converter 5 and is utilized to vary the input voltage applied to each addressable detection node 2. The level of the voltage input into the addressable detection nodes 2 is determined by empirical methods and varies depending on the particular DNA assay being analyzed.

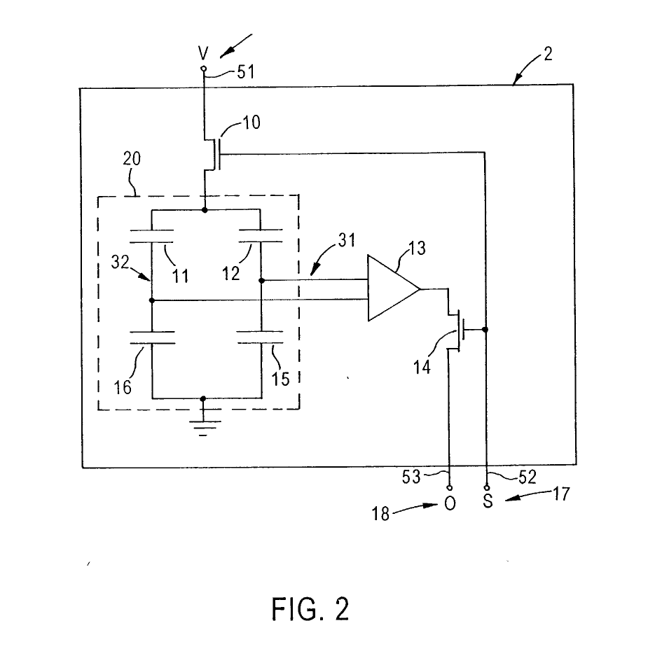

The second signal line is the select line 52, which as explained in conjunction with FIG. 2 is utilized to select a given addressable detection node 2 for analysis. As shown, each select line 52 is also coupled to the output of the node selection decoder 4. In the preferred embodiment, node selection decoder 4 functions to activate a single select line 52 at a time so as to allow for analysis of the corresponding detection node 2. The detection node 2 to be selected is determined by the digital control signal 6 coupled to the input of the node selection decoder 4. The digital control signal 6 is generated by a computer/controller (not shown). As stated, the node selection decoder 4 is utilized to select any one of the detection nodes 2 by inputting a signal corresponding to the address of the desired detection node 2 into the node selection decoder 4. During operation, for example, the selection node decoder 4 may be controlled so as to sequentially activate each detection node 2 so as to allow for determination of whether or not the known DNA and the unknown DNA in the given detection node 2 hybridized.

The third signal line 53 is an output line which functions to couple the output signal generated by the given detection node 2 to the A/D converter 3. As explained in further detail below, the output signal of a given detection node 2 exhibits a first voltage level if the known DNA and the unknown DNA in the given detection node 2 hybridize, and exhibits a second voltage level if the known DNA and the unknown DNA do not hybridize. The output line 53 of a given detection node 2 is activated upon selection of the given detection node 2 by the selection node decoder 4. Accordingly, in the preferred embodiment, only one output signal line 53 is active at a time. The output of the A/D converter 3 is coupled to a computer/controller (not shown) for analysis and recordation of the data. For example, assuming the voltage level of the output signal of detection node “X” indicates hybridization has occurred, as the identification of the known DNA deposited in detection node “X” is recorded and stored in memory, upon receiving the signal indicating hybridization has occurred, the computer/controller retrieves the data associated with detection node “X”, which identifies the known DNA contained in detection node “X”, and labels the unknown DNA placed in detection node 2 equal to the known DNA.

FIG. 2 is an exemplary schematic diagram illustrating one embodiment of a detection node 2, which is also referred to as a biosensor cell. Referring to FIG. 2, each biosensor cell 2 includes a voltage divider circuit 20 comprising capacitors 11, 12, 15 and 16, a differential amplifier 13 and transistors 10 and 14 which function to activate the given biosensor cell 2 and to couple the output of the biosensor cell 2 to the A/D converter 3. The operation of the biosensor cell 2 is as follows.

First, in order to select/activate a given biosensor cell 2, the corresponding selection line 52 must be made active by the selection node decoder 4. Activation of the selection line 52 functions to turn on transistors 10 and 14. As a result, the voltage signal “V” on the first signal line 51 is coupled to the voltage divider circuit 20 and the output of the differential amplifier 13 is coupled to the output signal line 53. The voltage divider circuit 20 essentially comprises two voltage divider circuits. The first divider circuit is formed by capacitors 11 and 16, and the second divider circuit is formed by capacitors 12 and 15. The first divider circuit generates a first reference voltage at node 32, which is coupled to one input of the differential amplifier 13, and the second divider circuit generates a second reference voltage at node 21, which is coupled to a second input of the differential amplifier 13.

Capacitors 11 and 12 are fixed capacitors and are fabricated utilizing standard semiconductor processing techniques and comprise, for example, a dielectric layer of silicon dioxide. Capacitor 16 is fabricated so as to allow for receipt of known DNA material, but not for receipt of unknown DNA material. The formation of the capacitor 16, which is detailed below, is such that the unknown DNA material functions as the dielectric component of the capacitor. Capacitor 15 is fabricated so as to allow for receipt of the known DNA material and the subsequent receipt of the unknown DNA material, and similar to capacitor 16, the DNA material supplied to capacitor 15 functions as the dielectric material. Exemplary structures of capacitors 15 and 16 are described in detail further below. However, it is noted that the exposure and/or non-exposure to the known DNA and the unknown DNA can be accomplished utilizing various capillary designs for transporting such material as is well known by those of skill in the art. Indeed, as the preferred embodiment of the present invention entails forming the detection system on a single semiconductor chip, in one embodiment, after formation of the capacitors and the other circuitry noted above, an additional layer is formed on the semiconductor structure which includes the necessary capillary design so as to allow unknown DNA to be delivered to capacitor 15. The known DNA would be placed in capacitors 15 and 16 of each biosensor cell 2 during the fabrication process prior to placement of the additional layer on the semiconductor structure.

In operation, when unknown DNA is supplied to capacitor 15, if the unknown DNA matches the known DNA sample already contained in capacitor 15, the known DNA sample hybridizes to the unknown DNA, which effectively increases (e.g., doubles the DNA contribution to the total dielectric) the dielectric constant of capacitor 15, thereby increasing the capacitance value of capacitor 15. As is known, the capacitance value of a capacitor increases proportionally with an increase in the dielectric constant of the dielectric material, which is formed in-part by the DNA material contained in the capacitor 15.

As a result, when a unknown DNA and a known DNA match in a given biosensor cell 2, the capacitance value of capacitor 15 increases resulting in a change in voltage at node 31. Specifically, the voltage at node 31 equals (V*C₁₅)/(C₁₅+C₁₂), where V is the voltage input via signal line 51, and C₁₅ and C₁₂ are the capacitive values of capacitors 15 and 12, respectively. This change in voltage at node 31 generates a difference between the voltage at node 31 and the voltage at node 32. This difference voltage is amplified by differential amplifier 13 and output to the A/D converter 3, and coupled to a data analyzer/computer (not shown) for processing. In particular, when the difference voltage exceeds some predetermined threshold value, the unknown DNA input into capacitor 15 is deemed to be the same as the known DNA contained in capacitor 16 of the same biosensor cell 2.

It is further noted that it is also possible for capacitor 16 to be fixed to an empirical value that is substantially close to capacitor 15 before hybridization. In such an embodiment, before applying the unknown DNA material, the difference in voltage at node 21 and node 32 would be measured utilizing the differential amplifier 13. Then during hybridization, a voltage change from the originally measured difference voltage can be utilized to indicate that hybridization has occurred.

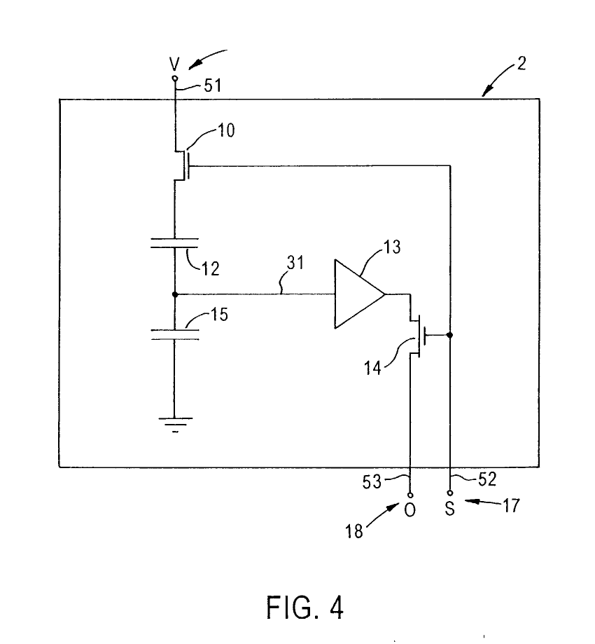

Another variation is to eliminate the first divider circuit formed by capacitors 11 and 16, such that only the second voltage divider circuit formed by capacitors 12 and 15 remain. In this embodiment, once the known DNA is supplied to capacitor 15, the voltage level at node 31 is measured and recorded. Then, once the unknown DNA is supplied to capacitor 15, the output of the amplifier is monitored to determine if a change in voltage level has occurred from the previously recorded level. If the change is larger than a predetermined amount, this indicates that hybridization has occurred. It is noted in both of these two variations, as explained above, the change in voltage would result from a change in the capacitance value of capacitor 15 as the result of hybridization. FIG. 4 illustrates a detection node formed in accordance with this variation. As shown, the detection node is substantially the same as that disclosed in FIG. 2, with the exception that the first divider circuit formed by capacitors 11 and 16 is omitted.

As noted above, in the preferred embodiment, capacitor 16 and capacitor 15 have a dielectric material comprising the known DNA, and capacitors 11 and 12 have the same capacitive values. As such, in the event there is no match between the unknown DNA and the known DNA, the voltage values at nodes 31 and 32 will be substantially equal, thereby generating a substantially zero voltage difference at the inputs to the differential amplifier 13. However, it is noted that it is also possible to design the system such that the capacitors have different values which generate different voltage levels at nodes 31 and 32 even when there is no hybridization. In such an embodiment, the data analyzer would be pre-programmed with the non-zero difference value (i.e., other than zero) input into the differential amplifier in the event no matched occurs. The data analyzer would then treat this non-zero difference value as the normal state (i.e., no match) and determine the occurrence of hybridization by judging the change in voltage relative to the non-zero difference value. It is further noted that the values of the capacitors 11 and 12 and the level of the input voltage “V” to the divider circuit necessary to accomplish the foregoing operations can be determined by empirical methods and will likely vary depending on the given DNA assay under consideration.

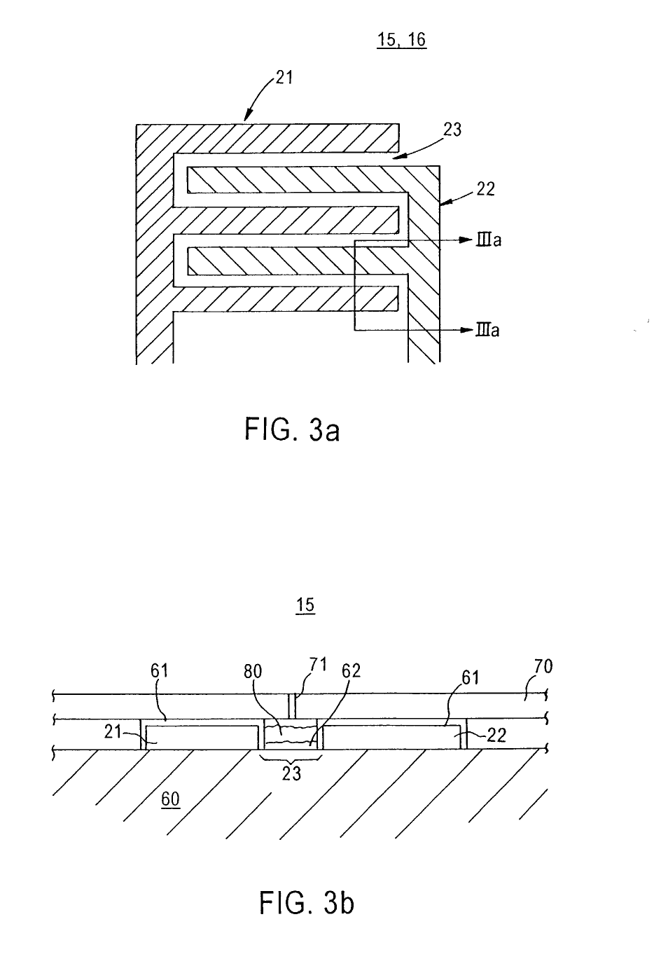

FIG. 3a illustrates a top view of an exemplary embodiment of a capacitor suitable for use as capacitors 15 and 16 illustrated in FIG. 2. Referring to FIG. 3a, the capacitor comprises an interdigitated layout having a first electrode 21 and a second electrode 22, both of which include a plurality of interdigitated fingers. The layout is such that each interdigitated finger is separated from the adjacent interdigitated finger by a predetermined space 23. As explained below with reference to FIG. 3b, space 23 functions to receive the DNA material which functions as the dielectric material of the capacitor.

FIG. 3b illustrates a cross-sectional view of the capacitor of FIG. 3a taken about lines IIIa-IIIa of FIG. 3a. As shown, the electrodes 21 and 22 are formed on an insulating layer 60. Each electrode is then coated with a film of a hydrophobic material 61 so as to prevent the DNA material from attaching to the metal electrodes 21 and 22. Further, the surface of the insulating layer 60 which is exposed via space 23 is coated with a hydrophilic material 62 so as to allow the binding of immobilization chemistry (i.e., the binding of the DNA material to the insulating layer 60 in space 23). It is noted that the hydrophobic material 61 also forms part of the dielectric layer of capacitors 15 and 16. Accordingly, in the preferred embodiment, it is desirable to deposit the hydrophobic material 61 evenly on electrodes 21 and 22 such that the dielectric constant attributable to the hydrophobic material 61 in capacitors 15 and 16 is equal. It is noted that it is desirable to maintain the intrinsic capacitance as low as possible.

The known DNA material 80 can be deposited in space 23 of capacitors 15 and 16 utilizing any of the known methods, which include, for example, a robotic spotter utilizing a standard pen device or ink jet technology. It is also noted that it is possible to deposit a different known DNA sample in each biosensor cell.

Once the known DNA is deposited in capacitors 15 and 16, a micro-machined manifold layer 70 is bonded to the upper surface of the semiconductor chip so as to enclose the capacitors 15 and 16. The micro-machined manifold layer 70 comprises a plurality of capillaries 71 (i.e., passageways), which allow for subsequent delivery of the unknown DNA sample to each capacitor 15 of each biosensor cell 2 in the array. It is noted that the capillaries do not provide access to capacitor 16 of any biosensor cell 2. The design of capillary networks capable of performing this function are known in the art.

Accordingly, in operation, the unknown DNA is supplied to capacitors 15 of each biosensor cell 2 via the capillary network. Once the unknown DNA is applied, the processing and operation of the apparatus is as set forth above.

As described above, the present invention provides significant advantages over the prior art. Most importantly, the method and system of detecting/identifying unknown DNA of the present invention allows for the elimination of the need for utilizing of an optical scanner during the detection process, and allows for real-time detection of unknown DNA. As such, the present invention allows for reduction in the overall cost and time associated with performing the detection analysis. In addition, the present invention allows for the elimination of the need for a optical florescent tag to be attached to the unknown DNA.

Another advantage of the present invention is that it eliminates the need for utilizing micromechanical devices in the detection system, thereby increasing the overall reliability of the detection system.

Yet another advantage is that the system of the present invention can be implemented in a single semiconductor integrated circuit chip, and the number of biosensor cells contained on the chip can vary from chip to chip depending on the intended application. Accordingly, a medical technician can simply supply the unknown DNA to the system chip and the system chip can determine if the unknown DNA matches any of the known DNA in the system chip without any further analysis or measurements being performed by the technician.

Numerous variations of the foregoing embodiments of the present invention are also possible. For example, a comparator can be included in each biosensor cell, which functions to generate an output signal only if the output of the differential amplifier is a above some predetermined level. In such an embodiment, the comparator functions to output a signal (e.g., logical “1”) only if hybridization occurs between the known DNA sample and the unknown DNA.

It is further noted that while the detection system of the present invention has been described with regard to identifying unknown DNA samples, it can also be utilized in conjunction with the identification of other chemical assays.

Although certain specific embodiments of the present invention have been disclosed, it is noted that the present invention may be embodied in other forms without departing from the spirit or essential characteristics thereof. The present embodiments are therefor to be considered in all respects as illustrative and not restrictive, the scope of the invention being indicated by the appended claims, and all changes that come within the meaning and range of equivalency of the claims are therefore intended to be embraced therein.

I claim:

1. An apparatus for identifying an unknown DNA sample, said apparatus comprising:
a plurality of detection nodes, each of said detection nodes operable for allowing an interaction between a known DNA sample and an unknown DNA sample, and for generating an output signal if hybridization occurs between said known DNA sample and said unknown DNA sample; and
a decoder operative for receiving an input signal indicative of which of said plurality of detection nodes should be selected for processing, and for outputting control signals which operate to activate said selected detection node;
wherein each of said detection nodes comprises a first capacitor having a capacitance value which varies if hybridization occurs between said known DNA sample and said unknown DNA sample contained in said detection node, said change in said capacitance value is operative for generating said output signal.

2. The apparatus of claim 1, wherein said output signal has an amplitude which varies in accordance with variations of said capacitance value of said first capacitor.

3. The apparatus of claim 1, wherein each detection node further comprises:
a first capacitive voltage divider circuit capable of generating a first reference voltage signal;
a second capacitive voltage divider circuit capable of generating a second reference voltage signal; and
a differential amplifier for receiving said first reference voltage signal and said second reference voltage signal as input signals, and for generating said output signal, said output signal representing a difference between said first reference voltage signal and said second reference voltage signal;
wherein said second capacitive voltage divider circuit includes said first capacitor, and a variation in said capacitance value of said first capacitor causes a corresponding variation in said second reference voltage signal.

4. The apparatus of claim 3, wherein said detection node further comprises:
a first pass transistor operative for coupling a voltage supply to said first capacitive voltage divider and said second voltage divider; and
a second pass transistor operative for coupling an output of said differential amplifier to an output port of said detection node;
said first pass transistor and said second pass transistor being activated by said control signals output by said decoder.

5. The apparatus of claim 1, wherein said first capacitor comprises said known DNA sample and is capable of receiving said unknown DNA sample.

6. The apparatus of claim 5, wherein hybridization of said known DNA sample and said unknown DNA sample causes an increase in said capacitance value of said first capacitor.

7. The apparatus of claim 3, wherein said first capacitive voltage divider circuit includes a second capacitor having said known DNA sample, said second capacitor comprising the same amount of the known DNA sample as said first capacitor such that said first reference voltage signal and said second reference voltage signal are equal if said known DNA sample and said unknown DNA sample do not hybridize in said first capacitor.

8. A biosensor cell for identifying an unknown DNA sample, said cell comprising:
a first capacitive voltage divider circuit capable of generating a first reference voltage signal;
a second capacitive voltage divider circuit capable of generating a second reference voltage signal; and
a differential amplifier receiving said first reference voltage signal and said second reference voltage signal as input signals, and for generating an output signal, said output signal representing a difference between said first reference voltage signal and said second reference voltage signal;
wherein said second capacitive voltage divider circuit includes a first capacitor having a known DNA sample disposed therein and capable of receiving said unknown DNA sample, said first capacitor having a capacitance value which varies if hybridization occurs between said known DNA sample and said unknown DNA sample when said unknown DNA sample is delivered to said first capacitor.

9. The biosensor cell of claim 8, wherein a variation in said capacitance value of said first capacitor causes a corresponding variation in said second reference voltage signal.

10. The biosensor cell of claim 8, wherein said output signal has an amplitude which varies in accordance with variations of said capacitance value of said first capacitor.

11. The biosensor cell of claim 8, further comprising:
a first pass transistor operative for coupling a voltage supply to said first capacitive voltage divider and said second voltage divider; and
a second pass transistor operative for coupling an output of said differential amplifier to an output port of said biosensor cell;
said first pass transistor and said second pass transistor being activated by external control signals.

12. The biosensor cell of claim 8, wherein hybridization of said known DNA sample and said unknown DNA sample causes an increase in said capacitance value of said first capacitor.

13. The biosensor cell of claim 8, wherein said first capacitive voltage divider circuit includes a second capacitor having said known DNA sample, said second capacitor comprising the same amount of the known DNA sample as said first capacitor such that said first reference voltage signal and said second reference voltage signal are equal if said known DNA sample and said unknown DNA sample do not hybridize in said first capacitor.

14. A biosensor cell for identifying an unknown DNA sample, said cell comprising:
means for generating a first reference voltage signal;
means for generating a second reference voltage signal; and
means for receiving said first reference voltage signal and said second reference voltage signal as input signals, and for generating an output signal, said output signal representing a difference between said first reference voltage signal and said second reference voltage signal;
wherein said means for generating said first reference voltage includes a first capacitor having a known DNA sample disposed therein and capable of receiving said unknown DNA sample, said first capacitor having a capacitance value which varies if hybridization occurs between said known DNA sample and said unknown DNA sample when said unknown DNA sample is delivered to said first capacitor.

15. The biosensor cell of claim 14, wherein a variation in said capacitance value of said first capacitor causes a corresponding variation in said first reference voltage signal.

16. The biosensor cell of claim 14, wherein hybridization of said known DNA sample and said unknown DNA sample causes an increase in said capacitance value of said first capacitor.

17. The biosensor cell of claim 14, wherein said means for generating a first reference signal includes a second capacitor having said known DNA sample, said second capacitor comprising the same amount of the known DNA sample as said first capacitor such that said first reference voltage signal and said second reference voltage signal are equal if said known DNA sample and said unknown DNA sample do not hybridize in said first capacitor.

18. A method of identifying an unknown DNA sample, said method comprising the steps of:
generating a first reference voltage signal;
generating a second reference voltage signal utilizing a first capacitor having a known DNA sample disposed therein and capable of receiving said unknown DNA sample, said first capacitor having a capacitance value which varies if hybridization occurs between said known DNA sample and said unknown DNA sample when said unknown DNA sample is delivered to said first capacitor; and
generating an output signal representing a difference between said first reference voltage signal and said second reference voltage signal.

19. The method of claim 18, wherein a variation in said capacitance value of said first capacitor causes a corresponding variation in said second reference voltage signal.

20. The method of claim 18, wherein hybridization of said known DNA sample and said unknown DNA sample causes an increase in said capacitance value of said first capacitor.

21. The method of claim 18, wherein said method of generating a first reference signal includes a second capacitor having said known DNA sample, said second capacitor comprising the same amount of the known DNA sample as said first capacitor such that said first reference voltage signal and said second reference voltage signal are equal if said known DNA sample and said unknown DNA sample do not hybridize in said first capacitor.
